(19) United States
(12) Patent Application Publication
(10) Pub. No.: US 2004/0151726 A1
(43) Pub. Date: Aug. 5, 2004

(54) Novel vaccine for prophylaxis and theraphy in vetirinary and human medicine

(76) Inventors: Vladimir Vrzal, Ivanovice na Hane (CZ); Libor Bittner, Vyskov (CZ); Evzen Weigl, Olomouc (CZ); Alois Rybnikar, Brno (CZ); Josef Chumela, Ivanovice na Hane (CZ)

Correspondence Address: Andrew S Marks, 47 Rice Road, Wayland, MA, 01778, US

(21) Appl. No.: 10/480,406

(22) PCT Filed: Jul. 29, 2002
(86) PCT No.: PCT/EP02/08435

(30) Foreign Application Priority Data
Jul. 27, 2001 (CZ) PV 2001-2732

(51) Int. Cl. A61K 39/00; A61K 39/38

(52) U.S. Cl. 424/184.1

(57) ABSTRACT

The present invention relates to a vaccine containing vaccination strains of dermatophytes, its use for immunoprophylaxis and for the treatment of dermaphytoses, as well as suitable methods for its preparation.

The present invention relates to a novel vaccine containing vaccination strains of dermatophytes, its use for immunoprophylaxis and for the treatment of dermaphytoses, as well as the method for its preparation. The vaccines of the invention can be used in both veterinary and human medicine.

At present, in veterinary medicine, a number of vaccines are used in both the prophylaxis and treatment of dermatophytoses. These are both live vaccines and inactivated vaccines which are described, amongst others, in CS 160324, PL 156844, SU 1734762, GB 2025222, SU 955570, SU 955571, SU 548947, SU 835446, RU 2013444, RU 2013445, EP 393371, DE 9218921 U1, PL 100958, U.S. Pat. No. 4,229,434, U.S. Pat. No. 5,277,904, U.S. Pat. No. 5,284,652, CS 201481, CS 246791, CS 261460, CZ 279278 and CZ 279982. All vaccines described in these publications have the parenteral application in common. Most of time, they are applied intramuscularly, rarely subcutaneously. As the parenteral application may possibly involve the danger of causing anaphylactic and anaphylactoid reactions in the organism that was vaccinated (Gudding R. and Naess B., Amer. J. Vet. Res. 47, 1986, 2415-2417, “Vaccination of cattle against ringworm caused by Trichophyton verrucosum”), an application of the vaccines produced so far has, of course, been ruled out in human medicine. As a consequence, above all, a vaccine which can also be used in human medicine is urgently required.

### DETAILED DESCRIPTION OF THE INVENTION

Firstly, the present invention relates to a novel vaccine containing vaccination strains of dermatophytes, its use for immunoprophylaxis and for the treatment of dermatophytoses.

The vaccines of the invention are applied in both human and veterinary medicine. The warm-blooded animals in veterinary medicine, which are to be treated according to the invention, particularly include domestic and productive livestock.

According to the invention, dermatophytosis is understood to be a disease of the skin and its adnexa caused by dermatophytes.

The invention relates to a vaccine containing at least one dermatophyte vaccination strain.

In this invention, dermatophytes include, amongst others, fungi of the genera Trichophyton, Microsporum, Epidermophyton, Arthroderma and Nannizzia.

Particularly preferred vaccination strains belong to the genus Trichophyton, in particular, the stains are preferred to be Trichophyton verrucosum CCM F-765 (a1), Trichophyton verrucosum CCM 8166 (a2), Trichophyton mentagrophytes CCM 8290 (a3) and Trichophyton rubrum CCM 8291 (a4). Cultures of the above-mentioned strains Trichophyton verrucosum CCM F-765 (a1) and Trichophyton verrucosum CCM 8166 (a2) were deposited by the applicants with the Czech Collection of Microogansim (CCM), Masaryk University, Tvrdeho 14, 602 00 Brno originally on 5 Nov. 1984 and 23 Feb. 1993, respectively, under the accession numbers CCM F-765 and CCM 8166, respectively. On 14 Dec. 1992 and 22 Jul. 2002, respectively, these depositions were changed into depositions according to the Budapest Treaty. Cultures of the above-mentioned strains Trichophyton mentagrophytes CCM 8290 (a3) and Trichophyton rubrum CCM 8291 (a4) were deposited there under the accession numbers CCM 8290 and CCM 8291, respectively, on 23 Jul. 2001 according to the Budapest Treaty.

A vaccine containing a combination of at least two of the above-mentioned vaccination strains of the genus Trichophyton is another preferred embodiment. A combination of three of the four above-mentioned vaccination strains is particularly preferred.

In this case, the ratio of the individual vaccination strains a1:a2:a3:a4 of 0 or 2-10:0 or 1-5:0 or 5-30:0 or 2-10, or 0 or 1-10:0 or 1-2:0 or 3-30:0 or 1-3, or 0 or 1-10:0 or 1-5:0 or 3-30:0 or 1-10 in the end product is particularly preferred.

For use in veterinary medicine, embodiments with a ratio a1:a2:a3 of 2:1:5 or 10:1:30 or the use of a1 (Trichophyton verrucosum CCM F-765) alone are particularly preferred.

For use in human medicine, embodiments with a ratio a1:a2:a3:a4 of 4:2:10:3 or 10:1:30:2 as well as the ratios a1:a3:a4 of 1:3:1 and a2:a4 of 1:2 or the use of a4 (Trichophyton rubrum CCM 8291) alone are particularly preferred.

Furthermore, it is preferred if the overall amount of the vegetative forms of the vaccination strains in the end product is at least 1 mio. per 1 ml. An overall amount of at least 5 mio. per 1 ml is more preferred, an overall amount of at least 10 mio. per 1 ml is most preferred.

The pH range of the end product is 3.0 to 10.0. In this case, suitable buffer systems, which the person skilled in the art is generally familiar with, such as e.g. acetate or carbonate buffer systems or pharmaceutically acceptable organic or anorganic acids such as citric acid, acetic acid or hydrochloric acid are used.

The preferred pH range is 6.0 to 8.0. In this case, phosphate buffer systems of various compositions which the skilled person is familiar with are used.

For use in human medicine, the overall content of formaldehyde is 0.02% or less and for use in veterinary medicine it is 0.05% or less.

This amount which remains after the inactivation is physiologically unproblematic and permissible by law. It has furthermore a disinfectant function and a function which supports the effects of prophylaxis and therapy. The formaldehyde can, if necessary, be supplemented or substituted by other substances. Apart from the particularly suitable formaldehyde, chemical measures or physical applications can be used such as, e.g. beta-propionolactone, binary ethylenimine and acetic acid.

An overall content of less than 0.02% or 0.05% formaldehyde is particularly preferred.

The vaccines of the invention can be applied parenterally, e.g. intramuscularly or subcutaneously or epicutaneously. For use in human medicine, the epicutaneous application, i.e. the application and distribution on the skin, is particularly preferred.

In particular, the epicutaneous application in the area between the toes, the main source of dermal mycoses of other parts of the human body is preferred. As has already been explained above the risk of possibly life-threatening anaphylactic or anaphylactoid reactions can be minimised by means of the epicutaneous application.

Moreover, the epicutaneous route of application involves further advantages since an injection is not necessary as would usually be the case with parenteral application of vaccines. Thus, as the patient does not suffer any pain or has less fear, the compliance is enhanced. The same, of course, also applies to veterinary medicine as the person carrying out the treatment does not have to expect a dangerous fear or pain reaction by the animals.

Another aspect of the invention is the provision of a method for producing the vaccine according to the invention against dermatophytoses, the method being described below:

The vaccination strains are propagated individually on agar media as culture mediums in suitable compositions which contain saccharide and organically bound nitrogen, optionally after adjusting the pH value in the meantime, at tempteratures ranging from 25 to 29° C. under sterile conditions for a period of 10 to 30 days until vegetative forms have been formed optionally. Then, the strains are homogenised in an aqueous 0.1% formaldehyde solution and the suspensions obtained are inactivated for at least 24 to 36 hours at a temperature of 18 to 26° C. The homogenisation process is carried out in such a way that the major part of the spores is separated from the mycelium without destroying their surface structure.

After determination of the number of vegetative forms, the individual strains are optionally mixed amongst each other and supplemented with a phosphate-buffered, optionally with an otherwise buffered physiological saline in such a way that the overall amount of the vegetative forms of all vaccination strains is at least 1 mio. per 1 ml, preferably at least 5 mio. per 1 ml vaccine, the pH is between 3 and 10, preferably between 6.0 and 8.0 and the content of formaldehyde present after inactivation is 0.02% and less in an application in human medicine and 0.05% and less in an application in veterinary medicine.

In this case, it is preferred that the mixture of the strains a1:a2:a3:a4 corresponds to the ratio of 0 or 2-10:0 or 1-5:0 or 5-30:0 or 2-10 in the end product, wherein the above-mentioned mixture ratios and individual strains are particularly preferred.

The antigenic composition of the vaccine of the invention is so broad that also immune reactions against homologous genera and cross reactions with other dermatophyte genera such as, e.g. Microsporum canis or Trichophyton equinum have been detected.

In the following, particularly preferred embodiments of the invention are described. This, however, only serves to illustrate but not to limit the invention.

### EXAMPLES OF PREPARATION AND APLLICATION

### Example 1

### Preparation of a Vaccine for Veterinary Medicine and its Application

For raising/propagating the vaccination strains of the genus Trichophyton, a culture medium was used which contained 1.2% agar, 5.0% saccharides and 0.3% organically bound nitrogen.

The culture medium was sterilised in a vapour autoclave for 20 to 30 minutes under an overpressure of 80 to 100 kPa. After adjusting the pH to a value of 6.0 to 7.0, the bottom of the flask was covered with the culture medium and the sterilisation was repeated under the aforementioned conditions. After solidification of the agar and control of the sterility, the culture flasks with the culture medium were inoculated individually with suspensions of the vaccination strains. For the inoculation, either lyophilised cultures or instantly prepared inoculated cultures of the vaccinations strains (CCM F-765, CCM 8166 and CCM 8290) were used. Every inoculated culture contained at least 0.5 mio. viable CFU per 1 ml. The inoculated cultures of the vaccination strains were distributed on the surface of the culture medium. The cultivation was carried out at a temperature of 25 to 29° C. for 10 to 30 days so as to achieve an optimum formation of vegetative forms. When the productivity of the fungus antigen was high, the cultures were removed from the surface of the culture medium and homogenised in an aqueous 0.1% formaldehyde solution. The homogenisation with the homogenizer was carried out in such a way that the major part of the spores is separated from the mycelium without the surface structure being destroyed. Aliqouts of the homogenous suspension formed were individually filled into storage containers and stored for inactivation of the vaccination strains at a temperature of 18 to 26° C. for 24 hours. Then, in a Bürker counting cell chamber, the number of vegetative forms was determined for the individual vaccination strains. It amounted to 41 mio. in 1 ml vaccine. The individual vaccination strains were mixed in such a way that, in the finished vaccine preparation, the ratio of Trichophyton verrucosum CCM F-765:Trichophyton verrucosum CCM 8166:Trichophyton mentagrophytes CCM 8290 was 2:1:5.

The content of formaldehyde was 0.05%.

By adding phosphate-buffered physiological saline, the pH of the preparation was adjusted to 6.0.

The vaccine prepared in this manner, was tested for sterility and the number of vegetative forms and was used for vaccinating cattle infected with trichophytosis. After two intramuscular vaccinations with a dose of 5 ml each, the disease in the cattle of two breeds was cured within 14 days after the revaccination.

In view of the results that have been achieved with immunobiological preparations used so far—here the therapy, as a rule, takes 1 to 3 months—the success was very surprising.

### Example 2

### Preparation of a Vaccine for Veterinary Medicine and its Application

For cultivating the vaccination strain, a culture medium having a share of agar of 1.3%, a saccharide content of 10.0% and a content of organically bound nitrogen of 0.5% was used. The culture medium was sterilised twice in a vapour autoclave for 30 minutes each under an overpressure of 120 kPa. The pH value of the culture medium was 6.6. This medium was inoculated with a suspension of the strain Trichophyton verrucosum CCM F-765, the cultivation temperature being maintained at 25-26° C. The culture was homogenised in a 0.1% formaldehyde solution after 15 days of cultivation. After a 36-hour inactivation at a temperature of 22° C., a phosphate-buffered physiological saline was added. The content of formaldehyde in the finished vaccine solution was 0.04%, the pH was 7.3 and the number of the vegetative forms of the vaccination strain was 22 mio. in 1 ml.

The vaccine solution was used in a cattle for testing and its protective effect was good.

### Example 3

### Preparation of a Vaccine for Veterinary Medicine and its Application

The preparation was conducted in a manner similar to Example 1. The ratio of the strains T. verrucosum CCM F-765, T. verrucosum CCM 8166 and T. mentagrophytes CCM 8290 was 10:1:30. The formaldehyde content was 0.04%, the pH was adjusted to 8.0, the overall number of the vegetative forms of all vaccination strains was 80.0 mio. in 1 ml vaccine.

The vaccine solution was tested for its tolerability in calves aged 1 month. 10 ml vaccine solution was administered intramuscularly into the gluteus muscle of five calves. 24 hours before the vaccination, at the time of the vaccination, 4 hours after the vaccination and on the following four days, the temperature in the calves was measured rectally. The vaccine was tolerable, the body temperature in all animals remained within the range of the physiological values. No undesired local or other after-effect reactions could be observed in the vaccinated calves.

### Example 4

### Preparation of a Vaccine for Human Medicine and its Application

The vaccination strains were propagated in a culture medium containing 1.5% agar, 15.0% saccharides and 1.0% organically bound nitrogen.

The culture medium was sterilised twice for 25 minutes each in a vapour autoclave under an overpressure of 80 to 100 kPa. Inoculation, cultivation of the vaccination strains, homogenisation, inactivation and determination of the number of vegetative forms were carried out in the same manner as described above in Example 1. The vaccination strains Trichophyton verrucosum CCM F-765, Trichophyton rubrum CCM 8291, Trichophyton mentagrophytes CCM 8290 were mixed at a ratio of 1:1:3.

The suspension obtained was mixed with a suitable amount of phosphate-buffered physiological saline so that the resulting content of remaining formaldehyde was 0.02% and the overall amount of the vegetative forms contained in the end product was 10 mio. Depending of the composition of the buffer solution, the pH was adjusted to values between 6.0 and 8.0.

After determining the number of vegetative forms, the formaldehyde content and the pH, the vaccine was tested in voluntary candidates for sterility and for harmlessness in humans. For this purpose, the vaccine was applied three times a week in an amount of 10 ml by rubbing it onto the skin between the toes of the lower extremities.

No harmful side-effects for humans could be detected.

Furthermore, the vaccine was applied twice epicutaneously on cattle that were infected with trichophytosis for experimental purposes. Compared to the non-vaccinated animals, the successful healing could be observed about one week earlier, which proves the therapeutic effectiveness of the vaccine of the invention that was prepared in this manner.

### Example 5

### Preparation of a Vaccine for Application in Human Medicine

The vaccination strain Trichophyton rubrum CCM 8291 was cultivated for 18 days at a temperature of 26-28° C. in a culture medium with a composition according to Example 2. The inactivation of the homogenised culture in a 0.1% formaldehyde solution at a temperature of 26° C. took 24 hours. After mixing the inactivated suspension with a phosphate-buffered physiological saline, the pH was 6.8, the formaldehyde content was 0.015% and the number of the vegetative forms of the vaccination strain amounted to 11 mio. in 1 ml.

A challenge test was conducted in calves with the vaccine solution. The calves that were vaccinated twice epicutaneously were sufficiently protected against an experimental trichophytosis infection as compared to the control animals that were not treated with the vaccine.

### Example 6

### Preparation of a Vaccine for Application in Human Medicine

The vaccine solutions were prepared from different amounts of the two dermatophyte strains Trichophyton verrucosum CCM F-765 and Trichophyton mentagrophytes CCM 8290. For culturing them, culture medium containing 1.5% agar with 8.0% saccharides and 0.8% peptone was used. The culture was sterilised twice for 30 minutes each in a steam steriliser under an overpressure of 120 kPa. The culture media were inoculated individually with the suspensions of the vaccination strains mentioned having a content of 1.0 mio per 1 ml inoculum. After 10 days of inoculation at 25-27° C., the cultures were homogenised in a 0.1% formaldehyde solution at 10,000 rotations per minute for 2 to 3 minutes. Subsequently, the suspensions were kept in the dark for 72 hours at a temperature of 18° C. so as to inactivate the vaccination strains. The inactivation was detected by means of an incubation test on the culture medium. The inactivated suspension was diluted with a phosphate-buffered physiological saline in such a way that the content of formaldehyde was 0.018%, the pH was 7.17 and the number of vegetative forms of the vaccination strains was 15.8 mio in 1 ml.

The vaccine was tested in rabbits for harmlessness. After intramuscular injection of 3 ml vaccine solution into the pelvic muscles, no undesired local reactions or changes in the general condition could be observed in any rabbit.

### Example 7

### Preparation of a Vaccine for Application in Human Medicine

The vaccination strains T. verrucosum CCM 8166 and T. rubrum CCM 8291 were cultured on a culture medium with a composition of 1.2% agar, 10.0% saccharide and 0.5% peptone. These substances were sterilised in an autoclave in water under the same conditions as in Example 6. The cultivation at 28-29° C. was concluded after 30 days, the cultures taken were homogenised in an 0.1% formaldehyde solution and inactivated for 24 hours at 24° C. The ratio of the strains T. verrucosum CCM 8166 and T. rubrum CCM 8291 present in the vaccine was 1:2.

The pH was adjusted to between 6.0 and 8.0 by adding phosphate-buffered physiological salines having different sodium hydrogen phosphate contents. The number of the vegetative forms was 5 mio. in 1 ml vaccine solution.

The vaccine solution was applied epicutaneously onto an area of 10×10 cm of sheared and scarified skin of three calves at a volume of 5 ml per animal on three consecutive days. Neither local nor other post-vaccination reactions could be observed in the animals.

### Example 8

### Preparation of a Vaccine for Human Medicine and its Application

The culture medium was prepared according to Example 2. The inoculation, cultivation and homogenisation of the culture was carried out in analogy to Example 1. For preparing the vaccine, 4 vaccination strains were used in the following ratio: T. verrucosum CCM F-765: T. verrucosum CCM 8166: T. mentagrophytes CCM 8290: T. rubrum CCM 8291=4:2:10:3.

The overall amount of the vegetative forms of the vaccination strains was 75 mio. in 1 ml, the formaldehyde content was 0.015%.

The pH was adjusted to between 6.0 and 8.0 by adding phosphate-buffered physiological saline solutions with different shares in sodium hydrogen phosphate.

The vaccine solution was tested epicutaneously in two voluntary candidates at a volume of 2 ml in the area between the toes on 3 consecutive days. The vaccine was harmless and did not cause any undesired clinical changes, neither locally nor with regard to the general condition.

### Example 9

### Preparation of a Vaccine for Human Medicine and its Application

The vaccine solution was prepared as in Example 8, with the only difference that the ratio of the strains a1 a2:a3: a4 was 10:1:30:2 and the pH of the solution was 6.9.

For the therapy, the vaccine solution was applied to infected areas between the toes as well as to the big toe of the left foot with clinical changes that point to a mycosis; it was not possible to isolate the pathogen. By rubbing the vaccine solution into the infected skin once a day with a volume of 2 ml on three consecutive days, a healing of the changes in the skin could be observed 7 days after the third application.

As can undoubtedly be seen from the above explanations and Examples, the present invention is therefore a highly effective and, in addition, readily applicable vaccine for the immunoprophylaxis and treatment of dermatophytoses in veterinary and human medicine. In veterinary medicine, the vaccine of the invention can be applied both parenterally, in particular intramuscularly and subcutaneously, and epicutaneously. Furthermore, in human medicine, the vaccine of the invention is particularly preferred to be applied epicutaneously by suitable application, e.g. by spraying on the human skin with sterile spray ampoule.

Thus, for the first time, a vaccine for the epicutaneous application against dermatophytoses is provided for the use in human medicine. By the epicutaneous application, the risk of a possible life-threatening anaphylactic or anaphylactoid reaction is excluded.

We claim:

1. A vaccine containing at least one of the following vaccination strains
a1) Trichophyton verrucosum CCM F-765;
a2) Trichophyton verrucosum CCM 8166;
a3) Trichophyton mentagrophytes CCM 8290; and
a4) Trichophyton rubrum CCM 8291.

2. The vaccine according to claim 1 wherein the ratio of the individual vaccination strains a1:a2:a3:a4 in the end product is 0 or 2-10:0 or 1-5:0 or 5-30:0 or 2-10.

3. The vaccine according to claim 1 wherein the ratio of the individual vaccination strains a1:a2:a3:a4 in the end product is 0 or 1-10:0 or 1-2:0 or 3-30:0 or 1-3.

4. The vaccine according to claim 1, 2 or 3 wherein the ratio of the vaccination strains a1:a2: a3 in the end product is 2:1:5 or 10:1:30.

5. The vaccine according to claim 1, 2 or 3 wherein the ratio of the vaccination strains a1:a3:a4 in the end product is 1:3:1.

6. The vaccine according to claim 1, 2 or 3 wherein the ratio of the vaccination strains a2:a4 in the end product is 1:2.

7. The vaccine according to claim 1, 2 or 3 wherein the ratio of the vaccination strains a1:a2:a3:a4 in the end product 4:2:10:3 or 10:1:30:2.

8. The vaccine according to any one of the preceding claims characterised in that the overall amount of the vegetative forms of all vaccination strains is at least 1 mio. per 1 ml vaccine.

9. The vaccine according to any one of the preceding claims characterised in that the overall amount of the vegetative forms of all vaccination strains is at least 5 mio. per 1 ml vaccine.

10. The vaccine according to any one of the preceding claims characterised in that the overall amount of vegetative forms of all vaccination strains is at least 10 mio. per 1 ml vaccine.

11. The vaccine according to any one of the preceding claims characterised in that the pH of the end product is in the range of 3.0 to 10.0.

12. The vaccine according to any one of the preceding claims characterised in that the pH of the end product is in the range of 6.0 to 8.0.

13. The vaccine according to any one of the preceding claims containing less than 0.05% formaldehyde.

14. The vaccine according to any one of the preceding claims containing less than 0.02% formaldehyde.

15. The vaccine according to any one of the preceding claims for epicutaneous application.

16. The vaccine according to any one of the preceding claims for use in human medicine.

17. The vaccine according to any one of the preceding claims for use in veterinary medicine.

18. Use of at least one of the vaccination strains
a1) Trichophyton verrucosum CCM F-765
a2) Trichophyton verrucosum CCM 8166
a3) Trichophyton mentagrophytes CCM 8290; and
a4) Trichophyton rubrum CCM 8291
for the preparation of a vaccine for the immunoprophylaxis or treatment of dermatophytoses in humans or animals.

19. Use according to claim 18 of at least two of the vaccination strains.

20. Use according to claim 18 of at least three of the vaccination strains.

21. Use according to any one of claims 18 to 20, wherein the ratio of the vaccination strains is as in any one of claims 2 to 7.

22. Use according to any one of claims 18 to 21 wherein the vaccine is applied epicutaneously.

23. A method for the production of a vaccine against dermatophytosis comprising:
a) individually culturing the vaccination strains to be combined on agar media in a suitable composition as a culture medium, the agar media containing saccharide and organically bound nitrogen, optionally after intermediate adjustment of the pH at temperatures ranging from 25 to 29° C. under sterile conditions for a period of 10 to 30 days until vegetative forms are formed optimally,
b) subsequently homogenising in an aqueous 0.1% formaldehyde solution wherein the major part of the spores is separated from the mycelium without their surface structure being destroyed,
c) inactivating the strains in the suspension formed at a temperature of 18-26° C. for at least 24 to 36 hours,
d) adjusting the ratio between the vaccination strains,
e) adjusting the number of vaccination strains to at least 1 mio. in 1 ml finished preparation,
f) adjusting the pH to a range of 3.0 to 10.0, and
g) adjusting the remaining content of formaldehyde, optionally subsequently substituting the formaldehyde.
